(19) United States
(12) Patent Application Publication
(10) Pub. No.: US 2007/0250025 A1
(43) Pub. Date: Oct. 25, 2007

(54) COMPOSITE STRUCTURAL/THERMAL MAT SYSTEM

(75) Inventors: Matthew K. Sams, Apex, NC (US); Joseph A. Seiter, Raleigh, NC (US); Dwight S. Musgrave, Granville, OH (US)

Correspondence Address: BARNES & THORNBURG LLP, 11 SOUTH MERIDIAN, INDIANAPOLIS, IN, 46204, US

(73) Assignee: Martin Marietta Materials, Inc., Raleigh, NC (US)

(21) Appl. No.: 11/380,026

(22) Filed: Apr. 25, 2006

(51) Int. Cl. A61F 13/15

(52) U.S. Cl. 604/385.01

(57) ABSTRACT

A combination structural/thermal mat for use in environments subject to extreme low temperatures for at least a portion of a year comprises multiple portions including a structural portion, an insulation portion positioned vertically below the structural portion, and an energy-absorption portion positioned vertically below the insulation portion. The energy-absorption portion comprises a material that changes phase at a temperature below 0° C.

### FIELD OF THE DISCLOSURE

The present disclosure relates generally to high strength-to-weight support platforms and temporary roadbeds. More specifically, the present disclosure relates to temporary work platforms and road structures used in an environment subject to extreme cold.

### BACKGROUND OF THE DISCLOSURE

In some instances, a work site is located in a remote site with no established road leading to the site. Remote construction may require access of large equipment, but not require long-term access by road. When a permanent road is not required, a temporary road that is capable of supporting the traffic necessary to complete the construction may be utilized.

In some cases, a road to a work site is established by clearing trees and other obstacles and utilizing the bare ground as a roadbed. A temporary road may also be established by placing crushed stone on a path created within a remote area. In both of these cases, the ground provides structural support for equipment traveling on the temporary roadbed. In a site which is subject to precipitation, the quality of the road deteriorates with the presence of the precipitation creating muddy conditions, thereby causing long-term damage to the temporary road.

In some cases, a temporary road is established in an environment subject to extreme cold during a significant portion of the year. In this situation, the frozen ground provides a viable roadbed during the portion of the year in which the ground remains frozen. However, in some cases the surface of the ground is subject to thawing during at least a portion of the year creating muddy conditions and damage similar to the situations discussed above created by precipitation.

### SUMMARY OF THE DISCLOSURE

The present disclosure comprises one or more of the features recited in the appended claims and/or the following features which, alone or in any combination, may comprise patentable subject matter:

A mat is used to form a work surface such as a temporary road, for example. The mat comprises an energy-absorption portion and a structural portion positioned vertically above the energy-absorption portion. In some embodiments, an insulation portion may be interposed between the energy-absorption portion and the structural portion. The structural portion may be configured to support industrial traffic. The structural portion may comprise a fiber-reinforced polymer (FRP) panel, for example.

In some embodiments, the insulation portion may comprise urethane. The energy-absorption portion may comprise a material that changes phase at a temperature below 0° C. Exemplarily, the energy-absorption portion may include a solution of salt in water. The salt in solution may be chosen from the group of sodium chloride, calcium chloride, or magnesium chloride. In some embodiments, sodium chloride may be in a solution between about 15% and 25%. In some embodiments, the energy-absorption portion may comprise a solution of glycerin in water. In other embodiments, the energy-absorption portion may comprise a solution of alcohol in water. In still other embodiments, the energy-absorption portion may comprise a solution of ethylene glycol in water.

In some embodiments, the energy-absorption portion may be secured to the ground and the structural portion may be secured to the energy-absorption portion. In embodiments where the insulation portion is interposed between the energy-absorption portion and the structural portion, the insulation portion may be secured to the energy-absorption portion and the structural portion may be secured to the insulation portion. In some embodiments, a plurality of materials may be combined to create a phase changing material to keep the phase change temperature approximately constant between the first freezing and last freezing areas of the energy-absorption portion of the mat. For example, solutions containing mixtures of sodium chloride, magnesium chloride, calcium chloride, ethylene glycol, glycerin, or ethyl alcohol or combinations thereof may be used.

A method of using a mat comprises the steps of absorbing thermal energy with an energy-absorption portion of the mat so as to impede heat transfer through the mat to a portion of ground beneath the mat that is frozen, the energy-absorption portion comprising a material which changes phase at a temperature below 0° Celsius, and maintaining the portion of the ground beneath the mat frozen as a result of the absorbing step.

In some embodiments, the method includes the step of exposing the energy-absorption portion to a temperature below the phase change temperature of the material to induce at least a portion of the material to change phase. In some other embodiments, the method includes the step of insulating the energy-absorption portion with an insulation portion above the energy-absorption portion. In still other embodiments, the method includes the step of supporting industrial traffic with a structural portion above the energy-absorption portion. In some embodiments where the energy-absorption portion is insulated by an insulation portion, the method may include the step of supporting industrial traffic on the structural portion above the insulating portion.

Additional features, which alone or in combination with any other feature(s), including those listed above and those listed in the claims, may comprise patentable subject matter and will become apparent to those skilled in the art upon consideration of the following detailed description of illustrative embodiments exemplifying the best mode of carrying out the invention as presently perceived.

### BRIEF DESCRIPTION OF THE DRAWINGS

The detailed description particularly refers to the accompanying figures in which:

FIG. 1 is a perspective view of an oil drilling work site in a arctic environment, the work site and an access road formed from a system of combination structural/thermal mats placed on the bare ground and arranged in a matrix to form a work surface;

FIG. 2 is a fragmented cross-section of a first embodiment of combination structural/thermal mat placed on bare ground to form a work surface;

FIG. 3 is a fragmented cross-section of a second embodiment of a combination structural/thermal mat placed on bare ground to form a work surface;

FIG. 4 is a fragmented cross-section of a third embodiment of a combination structural/thermal mat placed on bare ground to form a work surface;

FIG. 5 is a fragmented cross-section of a fourth embodiment of combination structural/thermal mat placed on bare ground to form a work surface; and

FIG. 6 is a chart showing the relationship of ambient air temperature and the temperature of an upper surface of the mat of FIG. 2 to the temperature of a 12-inch thick block of ice below the mat.

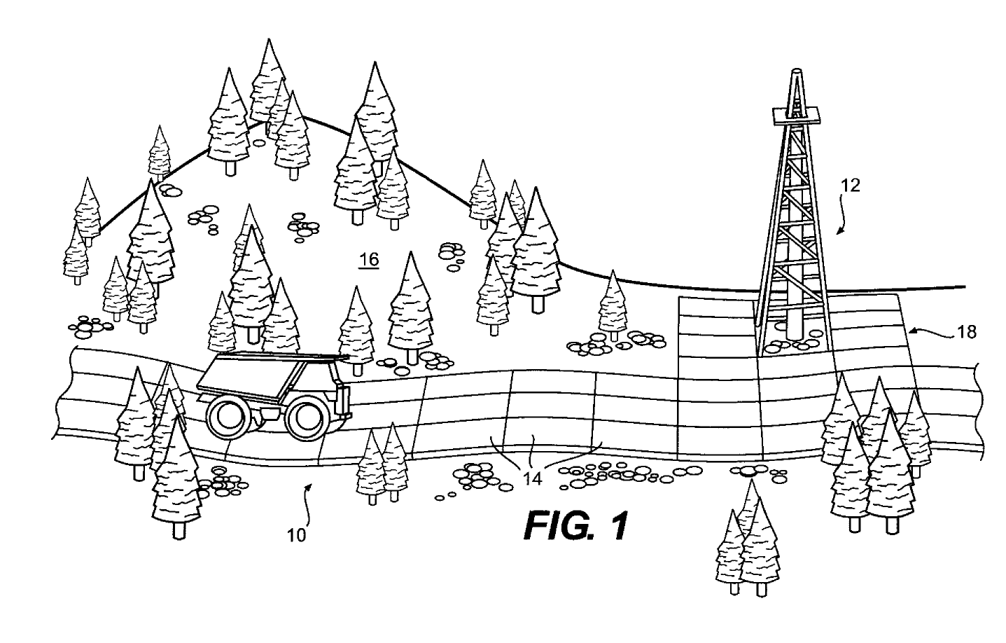

### DETAILED DESCRIPTION OF THE DRAWINGS

The present disclosure includes a temporary roadbed 10 leading to a work site 12 in an environment subject to extreme cold temperatures during a portion of a calendar year, such as an oil drilling work site in the arctic, for example. The temporary roadbed includes multiple mats 14 that are positioned on the ground 16. In addition, the mats 14 are positioned around the work site 12 to provide a work surface 18 for personnel and equipment to operate the work site 12. In the illustrative embodiment, the mat 14 is configured as a panel.

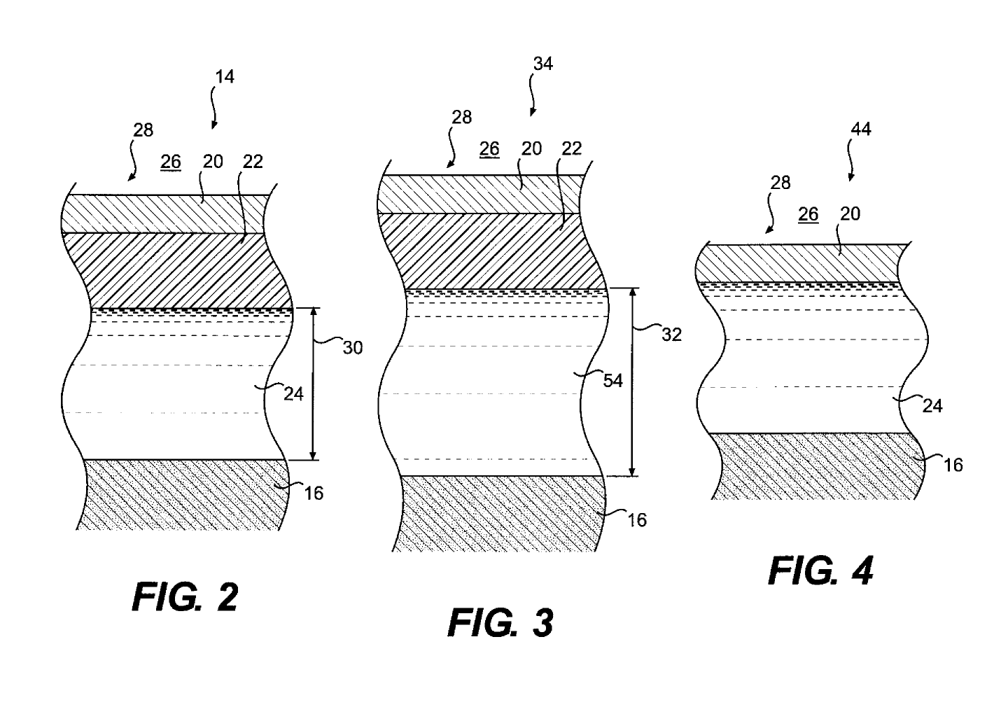

The mats 14 include multiple portions as shown in FIG. 2. An upper portion 20 is positioned vertically above an insulation portion 22. The insulation portion 22 is supported on an energy-absorption portion 24. The energy-absorption portion 24 is supported on the ground 16. Each portion 20, 22, 24 of the mat 14 is configured to support a load created by the personnel and equipment necessary to operate a remote work site. For example, the mats 14 are configured to support the load of a tractor-trailer vehicle.

The upper portion 20 is a structural member that supports and distributes the load across mat 14. The upper portion 20 may be embodied as one or more high-strength to weight composite panels. One exemplary type of composite panel is a fiber reinforced polymer (FRP) panel. Such an FRP panel may be formed of a polymer matrix composite material that includes a reinforcing agent and a polymer resin. The FRP panel may be embodied as any type of FRP structure. Examples of such structures include, but are not limited to, a solid laminate, a sandwich panel (e.g., a panel having upper and lower skins with a core therebetween), a pultruded or vacuum-fused panel, or a panel having upper and lower skins with vertical or diagonal webs therebetween. One type of a sandwich panel is TRANSONITE® available from Martin Marietta Materials, Inc. in Raleigh, N.C. Such a sandwich panel comprises upper and lower skins, a core therebetween and a plurality of fiber insertions extending from the upper skin through the core to the lower skin.

The matrix may include a thermosetting resin, although thermoplastic resins are also contemplated for use. Examples of thermosetting resins that may be used include, but are not limited to, unsaturated polyesters, vinyl esters, polyurethanes, epoxies, phenolics, and mixtures and blends thereof.

The reinforcing agent may include E-glass fibers, although other reinforcements such as S-glass, carbon, Kevlar, metal, high modulus organic fibers (e.g. aromatic polyamides, polybenzamidazoles, and aromatic polyimides), and other organic fibers (e.g. polyethylene and nylon) may be used. Blends and hybrids of the various reinforcing materials may be used. Other suitable composite materials may be utilized including whiskers and fibers such as boron, aluminum silicate, and basalt.

In the case of where the FRP panel is embodied as a sandwich panel, the core type may include, but is not limited to, balsa wood, foam and various types of honeycomb.

The FRP panel may be embodied as any of the structures disclosed in U.S. Pat. Nos. 5,794,402; 6,023,806; 6,044,607; 6,070,378; 6,081,955; 6,108,998; 6,467,118 B2; 6,645,333; 6,676,785, the entirety of each of which is hereby incorporated by reference. It should be appreciated that the structures disclosed in the above-identified patents may be sized, scaled, dimensioned, orientated, or otherwise configured in any desired manner to fit the needs of a given design of the FRP panel.

In the illustrative embodiment, the insulation portion 22 comprises urethane. The insulation portion 22 serves to impede the flow of heat from the upper portion 20. In simulation, an insulation portion 22 having conductivity of about 0.015 BTU-inch/hr.-ft.²-° F. was found to provide satisfactory results. Materials having varying conductive factors may be used depending on the thermal transfer requirements in the application.

It is a fundamental principle that heat flows whenever there is a temperature gradient in a system. Heat flows from areas of high heat to areas of low heat through the combination of conduction, convection and radiation. The thermal conductivity of a material can vary with temperature and thereby change the heat flux as temperature changes. In any open system, there is some level of heat flux as the system attempts to reach equilibrium. In the illustrative embodiment, heat is conducted from the upper portion 20 as the ambient temperature of the air 26 in contact with the upper portion rises above the temperature of the energy-absorption portion 24. In addition, an upper surface 28, of the upper portion 20 may be subject to radiation from the sun that tends to heat the surface 28. Heat from surface 28 may be transferred to the air 26 or conducted through the upper portion 20 to the insulation portion 22.

In the present invention, the mats 14 are constructed to take advantage of the ambient temperature changes in an arctic environment to control the flow of heat to the frozen ground below the mats 14. Due to seasonal changes, the air temperature varies from well below freezing to above freezing. In an environment, such as arctic regions for example, where the temperature stays below freezing for several months, heat flows from the ground through the mat 14 such that the mat 14 and ground beneath the mat 14 reach equilibrium with minimal heat flow therebetween. As the temperature above the mat 14 increases during the warmer months, heat tends to flow from the surface 28 of the upper portion 20 to the ground 16. A properly chosen insulation portion 22 has a low thermal conductivity and impedes the flow of heat from the surface 28 to the ground 16. Over time, the mat 14 will increase in temperature and heat will flow into the ground 16.

The mat 14 illustrative of the current invention minimizes the heat transferred from the surface 28 to the ground 16. The energy-absorption portion 24 of the illustrative embodiment acts as a heat sink and absorbs thermal energy to impede the heat transfer from the surface 28 to the ground 16. The energy-absorption portion 24 comprises a phase changing material that changes phase at a temperature below 0° C. In the illustrative embodiment, the energy-absorption portion 24 includes a solution of sodium chloride in water. The effect of the sodium chloride is to create a freezing mixture in the energy-absorption portion 24 when the temperature in the energy-absorption portion 24 falls below 0° C.

In a first mode, the energy-absorption portion 24 is charged as a heat sink. As the surface 28 of the upper portion 20 is exposed to ambient air that has a temperature lower than the temperature of the mat 14, heat flows from the energy-absorption portion 24 to the surface 28 where the heat is transferred away from the mat 14. As the temperature of the freezing mixture in the energy-absorption portion 24 is lowered, additional water freezes and the concentration of salt in the remaining solution is increased thereby depressing the freezing temperature of the remaining solution. The temperature of the freezing mixture continues to decline as more heat is removed from the mixture. The freezing mixture maintains a temperature that is slightly higher than the freezing temperature of the remaining solution, including both the ice in the mixture and the remaining solution.

In a second mode, heat is introduced to the surface 28 during warmer months by radiation from the sun and convection from ambient air having a temperature higher than the temperature of the mat 14. The heat flows from the surface 28 toward the ground 16. Heat flowing to the energy-absorption portion 24 begins to increase the temperature of the freezing mixture of the energy-absorption portion 24. To raise the temperature of the freezing mixture, a portion of the ice must undergo a phase change from solid to liquid.

It is known that the heat required to induce a phase change in a given mass of ice is much greater than the heat required to raise the temperature of an equivalent mass of water. Thus, the temperature in the energy-absorption portion 24 remains depressed as long as ice remains in the freezing mixture. The energy-absorption portion 24 consumes a considerable amount of energy in melting the ice and thereby maintains the temperature of the energy-absorption portion 24 below freezing. This maintains the ground 16 in communication with the energy-absorption portion 24 frozen and suitable for supporting the mat 14 without allowing the surface of the ground 16 to thaw and thereby reduces the chance the ground 16 will become muddy. In addition, the mat 14 prevents precipitation from reaching the ground 16 beneath the mat 14 and thereby prevents the precipitation from raising the temperature of the ground 16 or creating muddy conditions.

While the illustrative embodiment is disclosed utilizing a mixture of sodium chloride and water in the energy-absorption portion 24 it should be understood that multiple materials may be utilized in solution to keep the phase change temperature approximately constant between the first freezing and last freezing areas of the energy-absorption portion 24 of the mat. For example, solutions containing mixtures of sodium chloride, magnesium chloride, calcium chloride, ethylene glycol, glycerin, or ethyl alcohol or combinations thereof may be used.

The mat 14 is a self-contained unit such that multiple mats 14 may be positioned adjacent one another to form a road 10 or work surface 18. The mat 14 may include a connector to allow multiple mats 14 to be connected to form a structure such as a road 10 or work surface 18.

The portions 20, 22, 24 of the mat 14 are secured together through adhesive to form the mat 14. It should be understood that the portions 20, 22, 24 may be secured to one another through any of a number of the fasteners or securing methods. For example, the upper portion 20 may be secured to the insulation portion 22 through fasteners such as screws, nails, bolts, or the like. The upper portion 20 may also be secured to insulation portion 22 through other securing means such as welding, heat staking, or any of a number of other securing/adhering methods. The insulation portion 22 may be secured to the energy-absorption portion 24 in any of these approaches as well.

In some embodiments, the portions 20, 22, 24 may not be secured to one another, but positioned such that gravity maintains the relative position of the portions 20, 22, 24 to one another.

While in the illustrative embodiment, the mats 14 are self-contained units, in some embodiments, the various portions may be of varying sizes and the multi-layer structure may be constructed at the point of use by laying the energy-absorption portion 24 on the bare ground 16, positioning an insulation portion 22 on the energy-absorption portion 24, and positioning an upper portion 20 on top of the insulation portion 22.

The energy-absorption portion 24 of the illustrative embodiment contains the solution in a closed volume formed from a substantially watertight vinyl that is flexible to provide for the expansion of the volume when ice forms within the volume. It should be understood that any of a number of watertight materials might be used to form the energy-absorption portion 24.

Referring again to FIG. 2, a dimension 30 has been identified which represents the thickness of the energy-absorption portion 24 which is about 10 inches. In another embodiment of a mat 34 shown in FIG. 3, the thickness 32 of another embodiment of the energy-absorption portion 54 is about 14 inches, which is greater than the thickness 30 of the illustrative embodiment of mat 14. The thickness of mats 14, 34 may be varied depending on use conditions to create a larger heat-sink effect or to reduce the thickness of the energy-absorption portion 24 if the conditions of use minimize the need for the energy-absorption portion 24 during the warmer months.

In yet another embodiment, a mat 44 shown in FIG. 4 is similar to the mats 14 and 34, but the insulation portion 22 has been omitted. In some conditions, the insulation portion 22 may not be necessary and the upper portion 20 may be positioned directly on the energy-absorption portion 24. In other embodiments, the thickness of the insulation portion 22 may be varied to address particular application requirements.

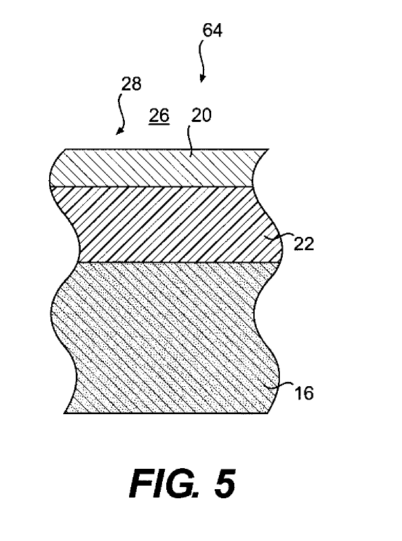

In still yet another embodiment, a mat 64 shown in FIG. 5 is similar to the mats 14, 34, and 44. In the mat 64, the energy-absorption portion 24 is omitted and the structural portion 20 is positioned on an insulation portion 22, which is supported on the ground 16. In certain applications, the insulation portion 22 may be configured to maintain the ground 16 in a frozen condition throughout the preferred work season, and therefore the energy-absorption portion 24 is unnecessary.

In the illustrative embodiments of mats 14, 34 and 44, the energy-absorption portion 24 comprises a solution of about 15% sodium chloride in water. It should be understood that the percentage of sodium chloride may be varied to adjust the mass of water that undergoes the phase change between ice and liquid at a given temperature. In still other embodiments, a solution of greater than about 15% such as a solution of about 23% may be used if the phase change temperature is to be minimized.

While the illustrative embodiment utilizes a solution of sodium chloride in water, in other embodiments any of a number of phase changing materials may be used in the energy-absorption portion 24. For example, solutions containing mixtures of sodium chloride, magnesium chloride, calcium chloride, ethylene glycol, glycerin, or ethyl alcohol or combinations thereof may be used.

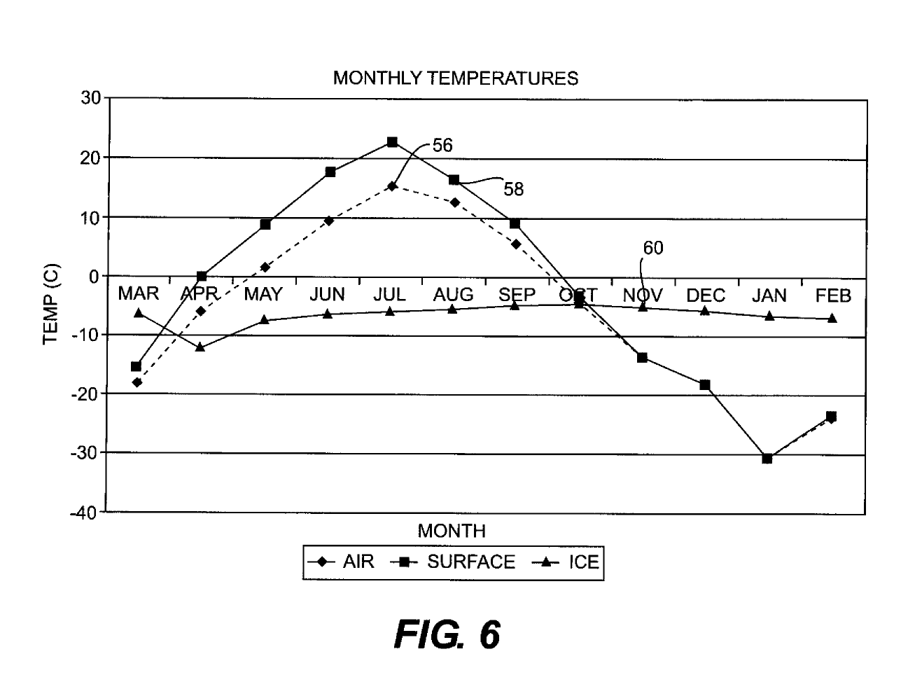

In one simulation, the results of which are shown in FIG. 6, the illustrative embodiment was modeled based on yearly climatic data from Tuktoyaktuk, Canada which is inside the Arctic Circle. The convection coefficient for the model was based on the assumed wind speed in the given month. The heat flux to the surface was based on solar radiation in the given month. The variables were chosen for a warm year. The model was based on the assumption that the mat 14 was placed on a 12-inch thick portion of ice. The air temperature 56 on the chart of FIG. 6, varied over the course of the year. Likewise, the temperature 58 at surface 28 varied of the course of the year based on the changes in solar radiation. The ice temperature 60 was simulated as the dependent variable. As can be seen from FIG. 6, the varied from about −12° C. to about −4° C. so that the ice remained frozen over the course of the year. Thus, the simulation confirms that in an arctic environment, the ground beneath the energy-absorption portion can be maintained in a frozen state throughout the course of a year.

There are a plurality of advantages of the present disclosure arising from the various features of the apparatus and methods described herein. It will be noted that alternative embodiments of the apparatus and methods of the present disclosure may not include all of the features described yet still benefit from at least some of the advantages of such features. Those of ordinary skill in the art may readily devise their own implementations of an apparatus and method that incorporate one or more of the features of the present disclosure and fall within the spirit and scope of the present disclosure.

1. A mat, comprising:
a structural portion configured to support industrial traffic, and
an energy-absorption portion positioned beneath the structural portion and configured to absorb thermal energy and impede heat transfer to a portion of ground beneath the mat, the energy-absorption portion comprising a material which changes phase at a temperature below 0° Celsius.

2. The mat of claim 1, further comprising an insulation portion interposed between the structural portion and the energy-absorption portion.

3. The mat of claim 2, wherein the insulation portion comprises urethane.

4. The mat of claim 2, wherein the insulation portion is secured to the energy-absorption portion and the structural portion is secured to the insulation portion.

5. The mat of claim 1, wherein structural portion comprises a fiber-reinforced panel.

6. The mat of claim 1, wherein the energy-absorption portion comprises a solution of sodium chloride in water.

7. The mat of claim 6, wherein the solution comprises about 15% to about 25% sodium chloride.

8. The mat of claim 1, wherein the material comprises glycerin in solution with water.

9. The mat of claim 1, wherein a plurality of mats are arranged to form a work surface.

10. The mat of claim 1, wherein the energy-absorption portion is configured to maintain the ground beneath the mat in a frozen state for at least two weeks after the adjacent ground has thawed.

11. A mat comprising an energy-absorption portion for absorbing thermal energy, the energy-absorption portion comprising a material which changes phase at a temperature below 0° Celsius.

12. The mat of claim 11, wherein the energy-absorption portion comprises a solution of sodium chloride in water.

13. The mat of claim 12, wherein the solution comprises about 15% to about 25% sodium chloride.

14. The mat of claim 11, wherein the material comprises glycerin in solution with water.

15. The mat of claim 11, wherein a plurality of mats are arranged to form a work surface.

16. A method of using a mat, comprising the steps of:
absorbing thermal energy with an energy-absorption portion of the mat so as to impede heat transfer through the mat to a portion of ground beneath the mat that is frozen, the energy-absorption portion comprising a material which changes phase at a temperature below 0° Celsius, and
maintaining the portion of the ground beneath the mat frozen as a result of the absorbing step.

17. The method of using a mat of claim 16, further comprising the step of exposing the energy-absorption portion to a temperature below the phase change temperature of the material to induce at least a portion of the material to change phase.

18. The method of using a mat of claim 16, further comprising the step of insulating the energy-absorption portion with an insulation portion above the energy-absorption portion.

19. The method of using a mat of claim 16, further comprising the step of supporting industrial traffic with a structural portion above the energy-absorption portion.

20. The method of using a mat of claim 19, further comprising the step of insulating the energy-absorption portion with an insulation portion above the energy-absorption portion.
